(19) United States
(12) Patent Application Publication
(10) Pub. No.: US 2026/0345950 A1
(43) Pub. Date: Nov. 5, 2026

(54) VIRTUAL REALITY (VR) TECHNIQUES FOR PROVIDING THERAPEUTIC TREATMENTS

(71) Applicant: 3D THERAPY, LLC, Beverly, MA (US)

(72) Inventors: Daniel Greenwald, Beverly, MA (US); Herbert J. Greenwald, Barnstable, MA (US); Robert Leo Vanderlin, II, North Conway, NH (US)

(21) Appl. No.: 19/744,701

(22) Filed: Jul. 16, 2026

Related U.S. Application Data
(63) Continuation of application No. 18/737,206, filed on Jun. 7, 2024.

(51) Int. Cl. A61M 21/02; G06F 3/01; G10L 25/66; G16H 20/10; A61M 21/00

(52) U.S. Cl. CPC A61M 21/02; G06F 3/015; G10L 25/66; G16H 20/10; A61M 2021/0022; A61M 2021/0027; A61M 2021/005; A61M 2021/0088; A61M 2205/3303; A61M 2205/3375; A61M 2230/10

(57) ABSTRACT

Systems and methods disclosed herein relate generally to providing virtual reality (VR)-based therapeutic, user-specific treatment for substance use disorder (SUD). An exemplary method includes outputting, by a voice assistant of an audible output device of a VR device, a user prompt configured to trigger a dynamic session builder with a user experiencing SUD, analyzing, by the voice assistant, one or more vocal responses received from the user, inputting the one or more vocal responses into a generative AI model, generating, by the generative AI model based on the one or more vocal responses of the user received during the dynamic session builder, one or more immersive multimedia scenes and/or one or more audible statements, and outputting the one or more immersive multimedia scenes and/or the one or more audible statements.

### RELATED APPLICATIONS

This application is a continuation of U.S. Application No. 18/737,206 filed on Jun. 7, 2024, the entirety of which is incorporated by reference herein.

### FIELD OF THE INVENTION

The present disclosure generally relates to virtual reality (VR) therapeutic treatments, and particularly treating substance use disorder (SUD) using VR.

### BACKGROUND

SUD is a worldwide epidemic, causing over 100,000 deaths annually in the United States (US) and much societal distress. 46.8 million US citizens have had or currently suffer from SUD. For example, more than 500,000 people in the US are dependent on heroin, and opioid use disorder (OUD) (most dangerously, Fentanyl) is wreaking havoc with families and costing the US over 100 billion dollars annually. Drug use is also the leading contributor to death from injuries. Ten million people aged 12 or older have used opioids such as Percocet, Oxycontin, Fentanyl, or heroin. This is a world-wide catastrophe that has quadrupled in the last 20 years.

Urgent action is needed to stem the severe disruption, emotional upheaval, loss of life, criminal behavior, financial cost, and societal turmoil caused by this disorder. Yet, most addicted persons do not seek therapy – only 18% of adults. Then, about 40% of enrollees drop out of treatment. And those who are treated have many relapses. Severe withdrawal symptoms caused by discontinuing opioid use are among the reasons for these problems.

Further, there are very few treatments for SUD, and those treatments are not very successful. Relapses occur frequently after treatment and current treatments are unpleasant. The process of recovering is emotionally and physically painful, leading to patients avoiding treatment or leaving treatment before it is completed. A better therapy is urgently needed.

### SUMMARY

The present embodiments may relate to, inter alia, VR systems and methods that create completely bespoke immersive experiences that play on multiple dimensions: spatial, audio, visual, and/or haptic.

The present embodiments may employ a sophisticated blend of psychologically informed machine learning and natural language processing. The therapeutic process may begin by conducting a comprehensive analysis of the user's historical data, behavioral patterns, clinician notes, and biometric / electroencephalography (EEG) responses. The present embodiments may then dynamically generate immersive scenarios ensuring a tailored therapeutic experience for each user.

A generative artificial intelligence (AI) model may utilize a real time feedback loop to engage users in nuanced conversations, adapting its responses based on emotional cues detected through natural language and biometric feedback. This emotional intelligence may enhance the ability to provide empathetic and personalized therapeutic support.

The present embodiments may integrate a continuous feedback loop by interfacing with biometric sensors, such as EEG devices. This real-time data stream may assess physiological responses. The system and/or method may dynamically adjust the VR experience, modifying environmental stimuli, challenges, and rewards based on the user's emotional and neurological states in real-time. For instance, if stress indicators are detected, the generative AI model may dynamically introduce a relaxation scenario, adjusting visuals, sounds, and challenges to promote calmness.

The present embodiments may provide enjoyable, gamified scenarios using generative AI that combine user preferences, therapeutic objectives, and/or game mechanics to create dynamic, engaging, and positive environments. Users may navigate through challenges and storylines that metaphorically mirror their personal journey, promoting active participation and emotional resonance.

The generative AI model may reference the user's historical data and clinician notes, including past successful interventions and preferences. For instance, if a particular scenario or therapeutic approach has been effective in the past, the generative AI model may incorporate similar elements into new experiences, ensuring continuity and building on proven therapeutic strategies.

The generative AI model may understand how to prescribe immersive experiences that help patients overcome addiction. The generative AI model may suggest and create fully generative immersive experiences to help patients to cope with triggers, cravings, and importantly, the underlying psychological roots that led to their addiction.

A mobile app may function as an extension of the VR experience. The mobile app may leverage cloud-based storage to recall and synchronize the user's historical data. Using natural language processing, the mobile app may facilitate ongoing therapeutic conversations, seamlessly transitioning between VR and mobile platforms and ensuring that the therapeutic process remains uninterrupted and accessible.

For example, in one instance, a VR system configured to provide therapeutic, user-specific treatment for substance use disorder (SUD) may include
(1) a VR device comprising a display screen positioned proximate to, or within a viewable distance from, eyes of a user experiencing SUD, the VR device communicatively coupled to one or more processors and one or more input devices;
(2) an audible output device communicatively connected to the one or more processors;
(3) a voice assistant accessible by the one or more processors and comprising a generative artificial intelligence (AI) model, wherein the generative AI model is trained with training data indicative of one or more psychological causes or psychological triggers of SUD and further trained with a plurality of immersive multimedia scenes designed to treat respective urges or cravings corresponding to the one or more psychological causes or psychological triggers of SUD; and
(4) an application (app) comprising computing instructions stored on a memory communicatively coupled to the one or more processors, wherein the computing instructions, when executed by the one or more processors, are configured to cause the one or more processors to:
   (a) output, by the voice assistant through the audible output device, a user prompt configured to trigger dynamic session builder with the user,
   (b) analyze, by the voice assistant, one or more vocal responses received from the user having user data indicative of a user-specific psychological cause or psychological trigger of the user causing an SUD of the user,
   (c) input, into the generative AI model, the one or more vocal responses,
   (d) generate, by the generative AI model based on the one or more vocal responses of the user received during the dynamic session builder conversation session, one or more immersive multimedia scenes and/or one or more audible statements designed to reduce urges or cravings corresponding to the user-specific psychological cause or psychological trigger of the user, and
   (e) output the one or more immersive multimedia scenes on the display screen and/or the one or more audible statements via the audible output device, to the user as a therapeutic treatment for the SUD. The system may include additional, less, or alternate functionality, including that discussed elsewhere herein.

In another aspect, a computer-implemented method for providing therapeutic, user-specific treatment for SUD. The computer-implemented method may be implemented via one or more local or remote processors, servers, memory units, mobile devices, laptops, desktops, smart watches, and/or other electronic or electrical components, which may be in wired or wireless communication with one another. For example, in one instance, the computer-implemented method may include:
(1) outputting, by a voice assistant of an audible output device of a VR device, a user prompt configured to trigger a dynamic session builder with a user experiencing SUD,
   (a) the VR device comprising a display screen positioned proximate to, or within a viewable distance from, eyes of the user, the VR device communicatively coupled to one or more processors and one or more input devices,
   (b) the audible output device communicatively connected to the one or more processors of the VR device, and
   (c) the voice assistant accessible by the one or more processors and comprising a generative artificial intelligence (AI) model, wherein the generative AI model is trained with training data indicative of one or more psychological causes or psychological triggers of SUD and further trained with a plurality of immersive multimedia scenes designed to treat respective urges or cravings corresponding to the one or more psychological causes or psychological triggers of SUD,
(2) analyzing, by the voice assistant, one or more vocal responses received from the user having user data indicative of a user-specific psychological cause or psychological trigger of the user causing an SUD of the user,
(3) inputting, by the one or more processors, the one or more vocal responses into the generative AI model,
(4) generating, by the generative AI model based on the one or more vocal responses of the user received during the dynamic session builder, one or more immersive multimedia scenes and/or one or more audible statements designed to reduce urges or cravings corresponding to the user-specific psychological cause or psychological trigger of the user, and/or
(5) outputting, by the display screen and/or the audible output device, the one or more immersive multimedia scenes and/or the one or more audible statements, to the user as a therapeutic treatment for the user’s SUD.

In addition, the systems and methods for providing therapeutic, user-specific treatment for SUD effect a particular treatment or prophylaxis for a disease or medical condition, i.e., SUD. The systems and methods provide a treatment that is particular: outputting immersive multimedia scenes and/or audible statements based on the vocal responses of the user that are designed to reduce urges or cravings. Providing the user with immersive multimedia and/or audible statements has been demonstrated effective for treating SUD. In research feasibility studies, a VR treatment system was tested with acute OUD detox patients. Data showed that the VR treatment system lessened opiate users’ overall discomfort and lessened urges and cravings within 10-to-15 minutes. Findings revealed a 74% improvement in depression, a 60% improvement in opioid cravings, a 70% improvement in anxiety, and importantly, a 40% increase in patient retention. The systems and methods further reduce the likelihood of users relapsing back into addiction and attract more people into seeking treatment for their SUD.

Further, improvements to the underlying computing system include the disclosed machine learning (ML) models, such as the generative AI model, which may receive feedback regarding their output and improve their predictions over time. In addition, the ML model(s) may be improved or updated via a dynamic session builder, which takes as input user voice data, and where the user voice data is used to update or transform the ML model (e.g., which be a type of large language model (LLM)) for use with the same and/or different users. The transformation and/or updates improve model’s ability to engage in various sessions with the same and/or different users based on the transformative feedback received from the user and/or various other difference users over time. Training data may be updated with new data over time, and the updated training data may be used to improve the ML model(s).

In addition, the present disclosure describes use of one or more particular machines, e.g., a VR device comprising a display screen and communicatively coupled to input device(s), an audible device, and/or one or more sensors, or otherwise devices or particular machines for the purpose of providing therapeutic, user-specific treatments for substance use disorder (SUD) as described herein.

Additional, alternate and/or fewer actions, steps, features and/or functionality may be included in an aspect and/or embodiments, including those described elsewhere herein.

### BRIEF DESCRIPTION OF THE DRAWINGS

The figures described below depict various aspects of the applications, methods, and systems disclosed herein. It should be understood that each figure depicts one embodiment of a particular aspect of the disclosed applications, systems, and methods, and that each of the figures is intended to accord with a possible embodiment thereof. Furthermore, wherever possible, the following description refers to the reference numerals included in the following figures, in which features depicted in multiple figures are designated with consistent reference numerals.

FIG. 1A illustrates an exemplary computer environment for providing VR therapeutic treatments.

FIG. 1B illustrates exemplary computing application modules method for providing VR therapeutic treatments.

FIGS. 2A and 2B illustrate a combined block and logic diagram in which exemplary computer-implemented methods and systems for training and operating generative AI models are implemented.

FIGS. 3A – 3D illustrate exemplary immersive multimedia environments.

FIGS. 4A and 4B illustrate an exemplary data flow for providing VR therapeutic treatments.

FIG. 5 illustrates an exemplary computer-implemented method for providing VR therapeutic treatments.

Advantages will become more apparent to those skilled in the art from the following description of the preferred embodiments which have been shown and described by way of illustration. As will be realized, the present embodiments may be capable of other and different embodiments, and their details are capable of modification in various respects. Accordingly, the drawings and description are to be regarded as illustrative in nature and not as restrictive.

### DETAILED DESCRIPTION

### FIELD OF THE DISCLOSURE

The systems and methods disclosed herein generally relate to, inter alia, generating and providing therapeutic treatment to users experiencing SUD. Some embodiments may include one or more of: outputting, by a voice assistant of an audible output device of a VR device, a user prompt, analyzing, by the voice assistant, one or more vocal responses received from the user, inputting the one or more vocal responses into a generative AI model, generating, by the generative AI model based on the one or more vocal responses of the user received during a dynamic session builder, one or more immersive multimedia scenes and/or one or more audible statements, and outputting, by a display screen and/or the audible output device, the one or more immersive multimedia scenes and/or the one or more audible statements, to the user as a therapeutic treatment for the user’s SUD.

As is commonly known and as used herein, VR refers to the use of any virtual environment, or mixed real-and-virtual environment, wherein at least a portion of human-to-machine or human-to-human interactions are generated using VR technology and/or VR devices. A VR environment may include one or more of augmented reality (AR), mixed reality (MR), extended reality (XR), or combinations thereof. A VR environment may include one or more visual environments or components, possibly with an audio component (e.g., spoken words of another person or a voice bot) or a text component as well. VR may refer to an immersive user experience, where the user can experience the sensation of a three-dimensional (3D) environment without real-world elements/scenes. AR may refer to an annotation, overlay, or augmentation of text or media content, such as graphics content, onto real-world content, such as images or video of a real-world scene, or onto a direct visual impression of the real world, such as may be seen through the transparent glass or plastic portion of smart glasses. MR may refer to an annotation, overlay, augmentation, or mixing of synthetic content, such as computer-generated graphics, virtual scenery, virtual images, or other mixed reality content with real-world content, such as real-world content.

A VR device may generally be any computing device capable of visualizing and presenting virtual content in conjunction with, or separate from, real-world content to generate a partial or wholly virtual environment or experience for a user. Exemplary VR devices may include a wearable AR, MR, or VR headset or smart glasses, smart contacts, smart displays or screens, a mobile device, a tablet, a device having a speaker and microphone, or a device having a text-based interface. A VR device may include one or more input controls, such as one or more physical buttons located on the VR device itself, or one or more physical buttons located on handheld controllers or devices worn on a hand, foot, or other body part (i.e., “worn devices”) used in conjunction with the VR device.

Handheld controllers or worn devices may include one or more inertia, orientation or position sensors to sense movements, gestures, positions, orientations, etc. of a wearer or user, or a body part of the wearer or user. For example, handheld controllers or worn devices may be used to virtually (e.g., using gestures) point at, select, activate, or otherwise interact with one or more elements of a UI provided or presented within a virtual environment via or using a VR device. Input may also be provided using physical touchscreen inputs on screens of the VR device (e.g., a screen of a smart phone or personal computer), or using a computing device (e.g., a smart phone or personal computer) associated with the VR device.

A VR device may also include audio or text input devices configured to enable a VR environment to include text-based interactions (e.g., virtual user interfaces within the virtual environment for selecting or otherwise entering text, and/or for presenting text), or audio (e.g., one or more speakers and one or more microphones of the VR device, to support spoken interactions). The audio and text input devices may also be configured to enable a wearer or user to interact with, respectively, a voice bot or a chatbot, for example. The audio and text input devices may also be used to generally control the VR device itself.

In some embodiments, a VR device and its input controls may be used to physically or virtually write text (e.g., using virtual gestures), type text (e.g., using a virtual or physical keyboard), and speak text.

In some embodiments, described VR devices may be any commercial VR device, such as a Google Glass® device, a Google Cardboard® device, a Google Daydream® device, a Microsoft Hololens® device, a Magic Leap® device, an Oculus® device, an Oculus Rift® device, a Gear VR® device, a PlayStation® VR device, an HTC Vive® device, and Apple Vision Pro®, to name a few. In general, each of these example VR devices may use one or more processors or graphic processing units (GPUs) capable of visualizing immersive multimedia scenes in a partial or wholly virtual environment.

For example, a Google Cardboard VR device may include a VR headset that uses one or more processors or GPUs of an embedded smart phone, such as a smart phone, which, in some embodiments, may be a Google Android-based or Apple iOS-based smart phone, or other similar computing device, to visualize immersive multimedia scenes in a virtual environment. Other VR devices, such as the Meta Quest VR device, may include a VR headset that uses one or more processors or GPUs of an associated computing device, such a personal computer/laptop, for visualizing immersive multimedia scenes in an VR environment. The personal computer/laptop may include one or more processors, one or more GPUs, one or more computer memories, and software or computer instructions for performing the visualizations, annotations, or presentation of immersive multimedia scenes or VR environments as described herein. Still further, VR devices may include one or more processors or GPUs as part of an VR device may operate independently from the processor(s) of a different computing device for the purpose of visualizing immersive multimedia scenes in a virtual environment.

A haptic feedback device may generally be any electromechanical device capable of providing tactile feedback to a user. The haptic feedback device may be a wearable device, such as gloves, vests, sleeves, and/or foot covers. The haptic feedback device may be handheld, such as a game controller or smartphone. The haptic feedback devices may provide tactile feedback through force, vibrotactile, electrotactile, ultrasonic, and/or thermal haptics. The described haptic feedback devices may be any commercial haptic feedback device, such as the bHaptics TactSuit® and TactGlove®, SenseGlove Nova®, and Manus Prime X Haptic VR®.

### EXEMPLARY COMPUTING ENVIRONMENT

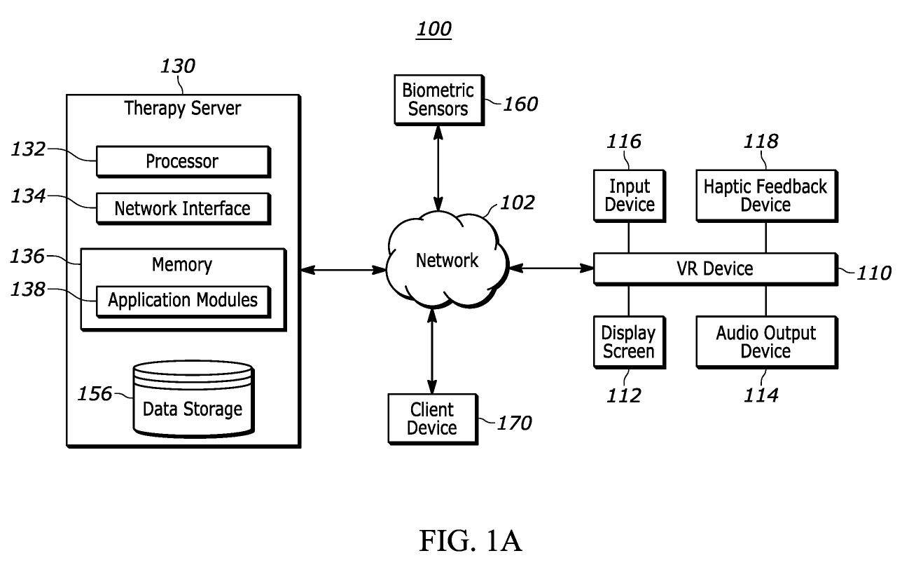

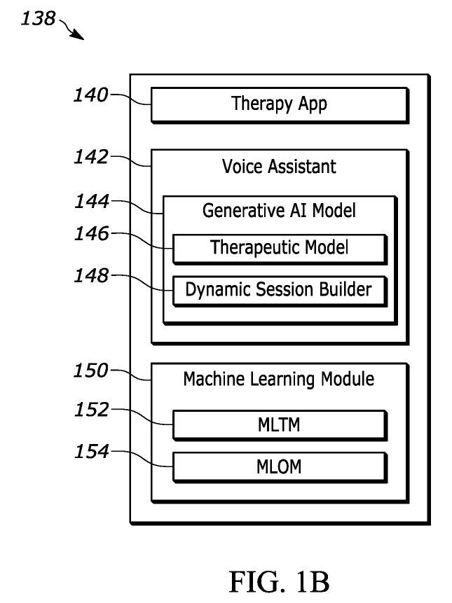

FIG. 1A depicts an example computing environment 100 in which techniques for VR therapeutic treatments are implemented. As illustrated in FIG. 1A, the computing environment 100 includes, in some embodiments, a network 102, a VR device 110, a therapy server 130, biometric sensors 160, and a client device 170. The computing environment 100 may include an on-premises computing environment, a multi-cloud computing environment, a public cloud computing environment, a private cloud computing environment, and/or a hybrid cloud computing environment. Although FIG. 1A depicts certain entities, components, equipment, and devices, it should be appreciated that additional or alternate entities, components, equipment, and devices are also possible.

The network 102 may include any suitable network or combination of networks, such as a local area network (LAN), a wide area network (WAN), the Internet, or a combination thereof. The network 102 may include a wireless cellular network (e.g., 4G, 5G, 6G, etc.). Generally, the network 102 enables bidirectional communication between VR device 110, therapy server 130, biometric sensors 160, and client device 170. The network 102 may comprise one or more routers, wireless switches, and/or other such wireless nodes communicating with the components of the computing environment 100 via wired and/or wireless communications based upon any one or more of various communications standards, including by non-limiting example, IEEE 802.11a/ac/ax/b/c/g/n (Wi-Fi), Bluetooth, and/or the like.

In one aspect, the VR device 110 may include a display screen 112 positioned proximate to, or within a viewable distance from, eyes of a user experiencing SUD. The display screen 112 may be configured to display two-dimensional (2D) or three-dimensional (3D) images and/or video. The VR device 110 may include one or more audio output devices 114. The audio output devices 114 may include speakers, headphones, or earbuds configured to output sounds to the user. The VR device 110 may include one or more one or more input devices 116. The input devices 116 may include a keyboard, mouse, trackpad, handheld controller, microphone, or any other suitable device for collecting user input. The VR device 110 may include one or more haptic feedback devices 118 for providing tactile feedback to a user.

In one aspect, the therapy server 130 includes a processor 132. In some embodiments, the processor 132 includes one or more central processing units (CPUs), graphics processing units (GPUs), field programmable gate arrays (FPGAs), and/or any other suitable processor. The processor 132 may be communicatively coupled to a memory 136 via a computer bus (not depicted) to create, read, update, transmit, delete, or otherwise access or interact with the data, data packets, or otherwise electronic signals to and from the processors 132 and memory 136, e.g., in order to implement or perform the machine-readable instructions, methods, processes, elements, or limitations, as illustrated, depicted, or described for the various flowcharts, illustrations, diagrams, figures, and/or other disclosure herein. The processor 132 interfaces with the memory 136 via a computer bus to execute an operating system and/or computing instructions contained therein, and/or to access other services/aspects. For example, the processor 132 interfaces with the memory 136 via the computer bus to create, read, update, delete, or otherwise access or interact with the data stored in the memory 136 and/or a data storage 156.

In one aspect, the therapy server 130 includes a network interface 134, which allows the therapy server 130 to communicate over the network 102 (e.g., with VR device 110, biometric sensors 160, and/or client device 170) via any suitable wired and/or wireless connection, e.g., using any suitable controller(s) of the network interface 134. In some embodiments, the network interface 134 includes one or more transceivers (e.g., WWAN, WLAN, and/or WPAN transceivers) functioning in accordance with IEEE reference standards, 3GPP reference standards, and/or other reference standards that receive and transmit data via external/network ports of the therapy server 130 connected to network 102.

In some embodiments, the memory 136 includes one or more memories and/or forms of volatile and/or non-volatile, fixed and/or removable memory, such as read-only memory (ROM), electronic programmable read-only memory (EPROM), random access memory (RAM), erasable electronic programmable read-only memory (EEPROM), and/or other hard drives, flash memory, MicroSD cards, and others. The memory 136 stores machine-readable instructions executable by the processor 132, including any of one or more application modules 138. The memory 136 also stores an operating system (e.g., Microsoft Windows, Linux, UNIX, etc.) capable of facilitating the functionalities, applications, methods, or other software as discussed herein.

In one aspect, the therapy server 130 includes and/or has access to (e.g., via network 102), the data storage 156. In some embodiments, the data storage 156 includes a relational database, such as Oracle, DB2, MySQL, a NoSQL based database, such as MongoDB, or another suitable database. The data storage 156 stores data and/or datasets for one or more users, such as previous session history and/or clinician notes, among other things. A dataset may include one or more types of data, records, files, etc. The terms “data” and “dataset” are used interchangeably herein.

In some embodiments, the computing environment 100 includes one or more biometric sensors 160 in contact with or in proximity of the user and configured to collect biometric data associated with a physiological state of the user. The biometric sensors 160 may include wearable devices, such as an Apple Watch, Fitbit Sense, Oura Ring, Circul+ Ring, or any other suitable device. The biometric sensors 160 may include dedicated medical devices, such as a blood pressure monitor, an electrocardiogram (ECG), or any other suitable device. The biometric data collected by the biometric sensors 160 may include heart rate, respiratory rate, electroencephalogram (EEG) data, galvanic skin response, pupil size, and/or any other suitable metric.

In one aspect, the client device 170 may include, by way of example, a tablet computer, a personal digital assistant (PDA), a mobile device smartphone also referred to herein as a “mobile device,” a laptop computer, a desktop computer, a portable media player, a wearable computing device, a virtual reality headset, smart glasses, a smart watch, a phablet, another smart device, a device configured for wired or wireless RF (Radio Frequency), etc. Of course, any network-enabled device appropriately configured may interact with the computing environment 100. The client device 170 may communicate with the network 102 via wired or wireless signals and, in some instances, may communicate with the network 102 via an intervening wireless or wired device, which may be a wireless router, a wireless repeater, a base transceiver station of a mobile telephony provider, an optical communications device, etc. The client device 170 may be owned and/or operated by a user. In one aspect, the client device 170 may be used to access therapy functionality when the VR device 110 is unavailable.

The client device 170 may include one or more processors, a memory, and other components not shown in FIG. 1A (e.g., a display, a communication unit, a user-input device, etc.), all of which may be interconnected via an address/data bus. The memory may include an operating system, a data storage, a plurality of software applications, and/or a plurality of software routines. The operating system, for example, may include one of a plurality of mobile platforms such as the iOS®, Android™, Palm® webOS, Windows Mobile/Phone, BlackBerry® OS, or Symbian® OS mobile technology platforms, developed by Apple Inc., Google Inc., Palm Inc. (now Hewlett-Packard Company), Microsoft Corporation, Research in Motion (RIM), and Nokia, respectively.

### EXEMPLARY APPLICATION MODULES

FIG. 1B depicts exemplary application modules 138 for implementing for VR therapeutic treatment techniques for SUD, such as OUD. As illustrated in FIG. 1B, the application modules 138 include, in some embodiments, a therapy application (app) 140, voice assistant 142, and a machine learning (ML) module 150.

In one aspect, the therapy app 140 includes computing instructions that enable a user and/or a clinician to receive and/or configure VR therapeutic treatment. The therapy app 140 may output, by the voice assistant 142 through the audio output device 114, a user prompt configured to trigger a dynamic session with the user. The user prompt may be a user-specific statement or question based on the user-specific data of the user or the one or more existing dynamic sessions with the user. The therapy app 140 may generate and output the user prompt to continue at least one of the one or more existing dynamic sessions with the user. The therapy app 140 may analyze, by the voice assistant 142, one or more vocal responses received from the user having user data indicative of a user-specific psychological cause, e.g., a psychological mechanism, or psychological trigger of the user causing an SUD of the user. The therapy app 140 may input, into the generative AI model 144, the one or more vocal responses. The therapy app 140 may generate, by the generative AI model 144 based on the one or more vocal responses and/or biometric data of the user received during the dynamic session, immersive multimedia scenes, and/or one or more audible statements designed to reduce urges or cravings corresponding to the user-specific psychological cause or psychological trigger of the user. The immersive multimedia scene may depict a three-dimensional (3D) room. The 3D room may be customized for the user based on the one or more existing dynamic sessions of the user. The 3D room may include selections or areas in 3D space for beginning execution of immersive experiences for implementing the therapeutic treatment for the user’s SUD. The therapy app 140 may output the immersive multimedia scene via the display screen 112, the audio output device 114,and/or haptic feedback via the haptic feedback device 118 to the user as a therapeutic treatment for the user’s SUD. For example, the immersive multimedia scene may depict a visualization of the user’s breath comprising particle simulations, and the particle simulations may mimic a flow of air during inhalation and exhalation by the user.

In one aspect, the therapy app 140 may output one or more results of the therapeutic treatment of the user’s SUD. The one or more results may include a graphic, chart, or data of the biometric data of the user and/or a survey by the user generated by collecting additional vocal responses of the user and/or by collecting input from the user operating the input device 116. The user and/or clinician may interact with the therapy app 140 via the VR device 110, the client device 170, and/or directly from a terminal of the therapy server 130.

In one aspect, the voice assistant 142 is configured to receive spoken input from the user and provide spoken output to the user. The voice assistant 142 may be programmed to simulate human conversation, interact with users, understand their needs, generate content, and/or recommend an appropriate line of action with minimal and/or no human intervention, among other things. This may include providing the best response of any query that it receives and/or asking follow-up questions. The voice assistant 142 may include a commercial voice assistant, such as Apple Siri, Google Assistant, and Amazon Alexa, or a custom voice assistant. The voice assistant 142 may employ supervised or unsupervised machine learning techniques, which may be followed by, or used in conjunction with, reinforced or reinforcement learning techniques. The voice assistant 142 may receive the spoken input from input device 116 and/or another microphone. The voice assistant 142 may provide the spoken output via the audio output device 114 and/or one or more other speakers.

In one aspect, the voice assistant 142 may include a generative artificial intelligence (AI) model 144. The generative AI model 144 may perform at least some of the functionalities and techniques disclosed herein, such as receiving user input, biometric data, previous session history, and/or clinical data and generating VR therapeutic sessions customized for the user. The generative AI model 144 may be an ensemble model that includes a plurality of sub-models, such as a therapeutic model 146 and a dynamic session builder model 148.

In one aspect, the application modules 138 include an ML module 150. The ML module 150 may include an ML training module (MLTM) 152 and/or an ML operation module (MLOM) 154. In some embodiments, at least one of a plurality of ML methods and algorithms may be applied by the ML module 150, which may include, but are not limited to: linear or logistic regression, instance-based algorithms, regularization algorithms, decision trees, Bayesian networks, cluster analysis, association rule learning, artificial neural networks, deep learning, combined learning, reinforced learning, dimensionality reduction, and support vector machines. In various embodiments, the implemented ML methods and algorithms are directed toward at least one of a plurality of categorizations of ML, such as supervised learning and reinforcement learning.

In one aspect, the ML based algorithms may be included as a library or package executed on the therapy server 130. For example, libraries may include the TensorFlow based library, the PyTorch library, the HuggingFace library, and/or the scikit-learn Python library.

In one embodiment, the ML module 150 employs supervised learning, which involves identifying patterns in existing data to make predictions about subsequently received data. Specifically, the ML model is “trained” (e.g., via MLTM 152) using training data, which includes example inputs and associated example outputs. Based upon the training data, the ML module 150 may generate a predictive function which maps outputs to inputs and may utilize the predictive function to generate ML outputs based upon data inputs. The exemplary inputs and exemplary outputs of the training data may include any of the data inputs or ML outputs described above. In the exemplary embodiments, a processing element may be trained by providing it with a large sample of data with known characteristics or features.

In yet another embodiment, the ML module 150 may employ reinforcement learning, which involves optimizing outputs based upon feedback from a reward signal. Specifically, the ML module 150 may receive a user-defined reward signal definition, receive a data input, utilize a decision-making model to generate the ML output based upon the data input, receive a reward signal based upon the reward signal definition and the ML output, and alter the decision-making model so as to receive a stronger reward signal for subsequently generated ML outputs. Other types of ML may also be employed, including deep or combined learning techniques.

The MLTM 152 may receive labeled data at an input layer of a model having a networked layer architecture (e.g., an artificial neural network, a convolutional neural network, etc.) for training the one or more ML models. The received data may be propagated through one or more connected deep layers of the ML model to establish weights of one or more nodes, or neurons, of the respective layers. Initially, the weights may be initialized to random values, and one or more suitable activation functions may be chosen for the training process. The present techniques may include training a respective output layer of the one or more ML models. The output layer may be trained to output a prediction, for example.

The MLOM 154 may comprise a set of computer-executable instructions implementing ML model loading, configuration, initialization and/or operation functionality. The MLOM 154 may include instructions for storing trained models. Once trained, the one or more trained ML models may be operated in inference mode, whereupon when provided with de novo input that the model has not previously been provided, the model may output one or more predictions, classifications, etc., as described herein.

### EXEMPLARY GENERATIVE AI MODELS

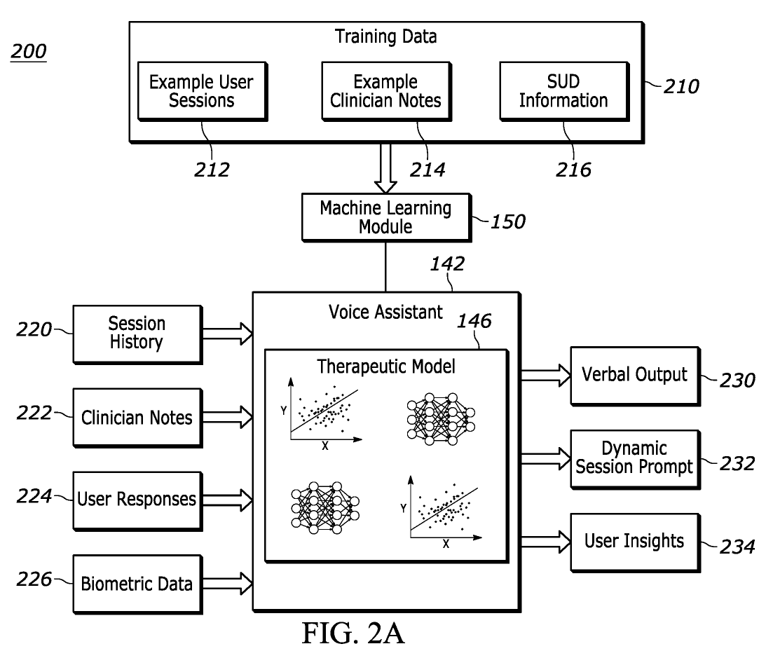

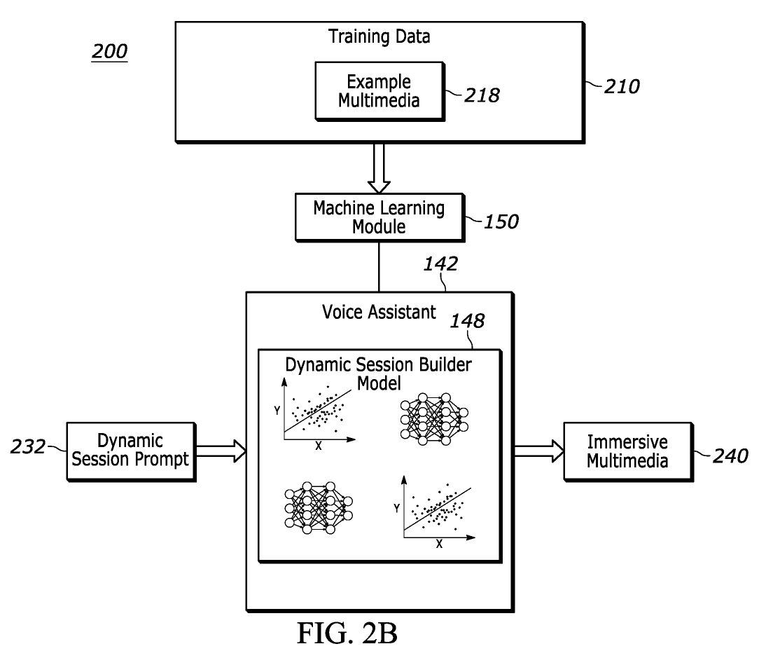

FIGS. 2A and 2B schematically illustrates how a generative AI model, including a therapeutic model 146 and dynamic session builder model 148, may be trained and operated. Some of the blocks in FIGS. 2A and 2B represent hardware and/or software components (e.g., block 150), other blocks represent data structures or memory storing these data structures, registers, or state variables (e.g., block 210), and other blocks represent output data (e.g., blocks 230, 232, 234, and 240). Input and output signals are represented by arrows.

An ML module 150 may include one or more hardware and/or software components, such as the MLTM 152 and/or the MLOM 154, to obtain, create, (re)train, operate and/or save one or more generative AI models 144, which itself may include the therapeutic model 146 and dynamic session builder model 148. To generate the therapeutic model 146 and/or the dynamic session builder model 148, the ML module 150 may use training data 210.

As described herein, the server, such as the therapy server 130, may obtain and/or have available various types of training data 210 (e.g., stored in the data storage 156). In an aspect, the training data 210 may labeled to aid in training, retraining, and/or fine-tuning the therapeutic model 146 and/or the dynamic session builder model 148. The training data 210 may include example user sessions 212, example clinician notes 214, SUD information 216, and/or example multimedia 218. The example user sessions 212 may include session transcripts and/or audio, user biometric data, and/or user feedback from one or more users. The example clinician notes 214 may include patient charts and/or clinician summaries of users. The example SUD information 216 may include descriptions of one or more psychological causes or psychological triggers of SUD and/or clinical texts describing psychological treatment of SUD. The example multimedia 218 may include music, narrative audio, images, and/or video designed to treat the psychological causes or psychological triggers of SUD. The example multimedia 218 may be labeled with a text description of the music, narrative audio, images, and/or video. The training data 210 may be in a structured or unstructured format. New training data 210 may be used to retrain or update the therapeutic model 146 and/or the dynamic session builder model 148. The dynamic session builder model 148 may be further trained with a plurality of immersive multimedia scenes. The therapeutic model 146 may be further trained with user-specific data of the user, such as previous dynamic sessions and/or clinical data.

While the example training data includes indications of various types of training data 210, this is merely an example for ease of illustration only. The training data 210 may include any suitable data that may indicate associations between model inputs and model outputs.

In an aspect, the server may continuously update the training data 210, e.g., based upon obtaining data sources related to newly user therapy sessions, or any other training data. Subsequently, the therapeutic model 146 and/or the dynamic session builder model 148 may be retrained/fine-tuned based upon the updated training data 210. Accordingly, the generation of verbal output 230, dynamic session prompts 232, user insights 234, and/or immersive multimedia scene 240 may improve over time.

In an aspect, the ML module 150 may process and/or analyze the training data 210 (e.g., via MLTM 152) to train the therapeutic model 146 to generate the verbal output 230, the dynamic session prompt 232, and/or the user insights 234. The therapeutic model 146 may be trained to generate the verbal output 230, the dynamic session prompt 232, and/or the user insights 234 via a neural network, deep learning model, Transformer-based model, generative pretrained transformer (GPT), although any type of applicable ML model/algorithm may be used, including training using one or more of supervised learning, semi-supervised learning, and/or reinforcement learning.

In an aspect, the ML module 150 may process and/or analyze the training data 210 (e.g., via MLTM 152) to train the dynamic session builder model 148 to generate the immersive multimedia scene 240. The dynamic session builder model 148 may be trained to generate immersive multimedia scene 240 via a neural network, deep learning model, Transformer-based model, generative pretrained transformer (GPT), although any type of applicable ML model/algorithm may be used, including training using one or more of supervised learning, semi-supervised learning, and/or reinforcement learning.

Once trained, the therapeutic model 146 and/or the dynamic session builder model 148 may perform operations on one or more data inputs to produce a desired data output. The therapeutic model 146 and/or the dynamic session builder model 148 may be loaded at runtime (e.g., by the MLOM 154) from a memory (e.g., the memory 136 of the therapy server 130).

The therapeutic model 146 may use generative AI to create unique conversations and experiences expertly designed to treat addiction. The therapeutic model 146 may learn about the patient based on a continuous feedback loop in a “conversation” and biometric measurements. Essentially therapeutic model 146 is getting to know the user. The therapeutic model 146 creates a bespoke version of an idyllic therapist based on Psychological Systems Theory and/or Mindfulness Based Cognitive Therapy. In one aspect, the therapeutic model 146 may process session history 220, clinician notes 222, user responses 224, and/or biometric data 226 inputs. The session history 220 may include information, including biometric data and therapies presented, from any prior sessions of the user. For example, the session history 220 may note that guided imagery has been effective for reducing this user’s stress in the past. The user responses 224 may include answers, responses, and/or feedback from the user in the current session. For example, the user may express, though natural language interaction, the need for relaxation. The biometric data 226 may include heart rate, respiratory rate, EEG data, galvanic skin response, pupil size, etc. For example, the biometric data 226 may indicate a physiological state, such as increased heart rate and blood pressure and a heightened state of alertness and stress.

The therapeutic model 146 may generate the verbal output 230, the dynamic session prompt 232, and/or the user insights 234. The verbal output 230 may include dialogue, questions, recommendations, and/or other speech for the user. For example, the verbal output 230 may include breathing exercises synchronized with the user's current state, gradually leading the user to a meditative and semi-hypnotic state. The verbal output 230 may embed therapeutic concepts and suggestions during this relaxed state to address underlying psychological factors contributing to the user’s substance use disorder. The verbal output 230 may include positive affirmations designed to counteract deeply ceded psychological roots. The dynamic session prompt 232 may include a prompt or other instructions for the dynamic session builder model 148. For example, the dynamic session prompt 232 may request that the dynamic session builder model 148 generate a serene virtual environment, such a peaceful beach setting. The therapeutic model 146 may guide the user through a guided imagery exercise, adjusting the scenario based on real-time physiological responses. The user insights 234 may include text summarizing a user session, providing a diagnosis, and/or recommending additional treatment.

In one aspect, the dynamic session builder model 148 may process the dynamic session prompt 232 input. The dynamic session builder model 148 may generate an immersive multimedia scene 240 as output. The immersive multimedia scene 240 may include virtual, stereoscopic scenes and images displayed by the VR device 110. The immersive multimedia scene 240 may include video, audio, images, and/or haptic feedback to treat the user. The dynamic session builder model 148 may generate the immersive multimedia scene 240 according to a preselected immersive experience theme. The preselected immersive experience theme may include a therapy session-based theme, a creative based theme, an action play motion theme, a meditation-based theme, an exploratory theme, a problem-solving skill building theme, or an interpersonal relationship building theme.

### EXEMPLARY IMMERSIVE MULTIMEDIA ENVIRONMENTS

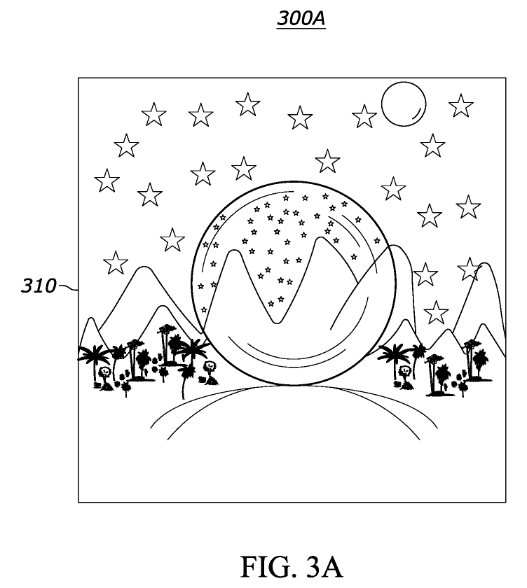

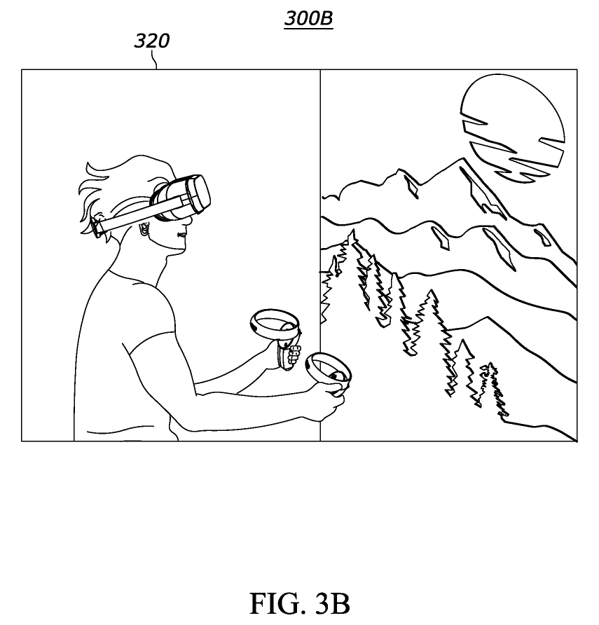

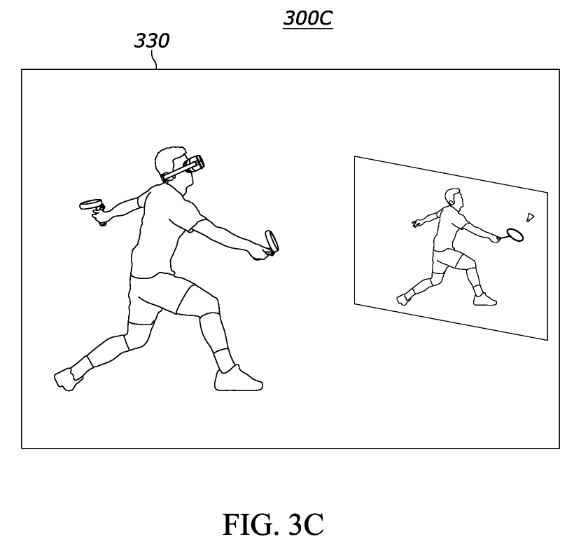

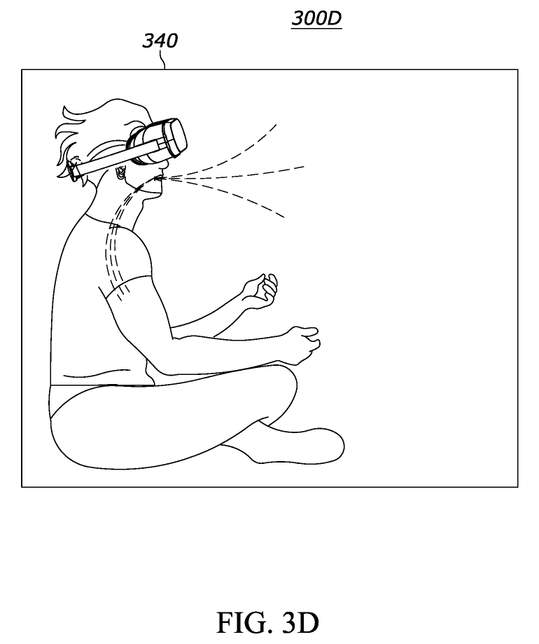

FIGS. 3A-3D depict exemplary immersive multimedia environments 300A-300D for implementing for VR therapeutic treatment techniques. The patient may begin the treatment in what appears to be a beautiful, comfortable virtual room. This room may serve as “home base” for the patient. In every way, it feels real to the user, including 3D audio. Over time the room may be customized to the patient’s specific needs – a “happy place” for that individual. From here, the patient will be guided to a wide variety of custom generative experiences.

In some embodiments, the generative AI model is trained to use motion, colors and patterns found in nature accompanied with haptic feedback and generative audio to promote a state of relaxation and calmness. Examples may include the calming sway of the leaves in a tree canopy, the mesmerizing patterns of the flight patterns of flocks of birds, as well as vibratory patterns that simulate a cascading sensation. During this aspect of the treatment the generative AI model is also trained to speak to the user in a very soothing, calming, positive manner. Unlike an actual person leading such an exercise, users appear not to feel judged while at the same time enjoying the sound of the voice output. If the generative AI model detects that a user is unusually stressed or anxious (via biometric data) it may create more experiences to increase feelings of well-being.

FIG. 3A illustrates an exemplary immersive multimedia environment 300A. The immersive multimedia environment 300A includes a non-human (so as to not pass judgment on the user) therapist orb 310 that converses with the user. The therapist orb 310 may have a knowledge of the user’s history and SUD treatment. The therapist orb 310 can be customized, e.g., gender, ethnicity, age, voice, tone, energy, etc., based on the user’s needs and preferences. All voices will be pleasant, calming and positive. This allows for a very comfortable and inviting interaction with the therapist orb 310. By being immersed in VR, the user may have a near-out-of-body experience, which creates a hypnotic, meditative state.

The patient may return to this room repeatedly and may engage in an in-depth therapy session at any time with the therapist orb 310. Through natural, spoken conversation with therapist orb 310, the AI system learns the user's name and personal preferences. The therapist orb 310 may ask questions to quantitatively assess the user’s level of discomfort and cravings. This helps inform and customize the immersive experiences and track progress towards goals over time. At the appropriate time the therapist orb 310 asks questions designed to gain an understanding of that patient’s specific psychological roots.

FIG. 3B illustrates an exemplary immersive multimedia environment 300B. The immersive multimedia environment 300B includes creative tools 320, such as painting or music tools, that allow the user to explore sound and/or make 3D drawings. VR allows the user to go beyond what is possible in the real world. The creative tools 320 may feel futuristic and intuitive and engage the user with music and colors. Drawing in 3D may allow the user to be expressive and use their entire body. The immersive multimedia environment 300B may seem like a private world in which the user feels no judgment in expressing himself or herself. Creativity has been shown to empower a state of mindfulness, but it also requires the user to overcome the sense that this may seem too difficult, or that they feel they are not “creative”. Through this highly immersive experience, even the simplest shape the user makes creates a huge sense of accomplishment, reinforced by the generative AI model’s dialogue. Users may have slight difficulty using the creative tools 320 at first and may have a natural tendency to give up. The immersive multimedia environment 300B allows them to problem solve with a low threshold for success (they easily feel successful). Any successful brush stroke is a win. Such experiences are shown to create a state of “flow” (as measured by biometric data) that is related to mindfulness and a positively altered sense of reality. In this environment, the generative AI model can begin to suggest changes to the user’s belief system that have contributed to their substance use. In such a state of mindfulness people are more open to suggestions that they would normally be resistant to accepting.

FIG. 3C illustrates an exemplary immersive multimedia environment 300C. The immersive multimedia environment 300Cmay include adaptive games 330 based on the user’s feelings and/or ability level. The adaptive games 330 may include active play and motion, such as capturing objects in VR, that engage the user with colors and music. If a user enjoys extreme activities, such as roller coasters, or skiing, the system can generate an experience that feels remarkably realistic, even simulating G-forces with the use of 3D environments, sound, and haptic feedback. The active play and motion may help release energy and increase feelings of well-being. The user may feel a lack of judgment in expressing himself or herself. The active play and motion provide quick ways to release energy.

FIG. 3D illustrates an exemplary immersive multimedia environment 300D. The immersive multimedia environment 300D includes conscious breathing and/or meditation 340 in VR. The conscious breathing and/or meditation 340 may bring calmness and peace to the user. The immersive multimedia environment 300D may be a hypnotic world, including music and color, in which the user may lose himself or herself in breath. The immersive multimedia environment 300D may be a calming environment that is generated based on the user’s needs and/or conscious breathing experience.

For example, the immersive multimedia environment 300D may visualize the breathing of the user in VR by generating custom, generative particle simulations that mimic the flow of air during inhalation and exhalation. Each individual requires different breath timing to achieve a state of mindfulness, meditation and extreme relaxation. The generative AI model may adjust the timing of the breathing of the user in real time and may be aware when the user has achieved the proper state by maintaining a feedback loop with the biometric measurements.

### EXEMPLARY DATA FLOW FOR PROVIDING VR THERAPY

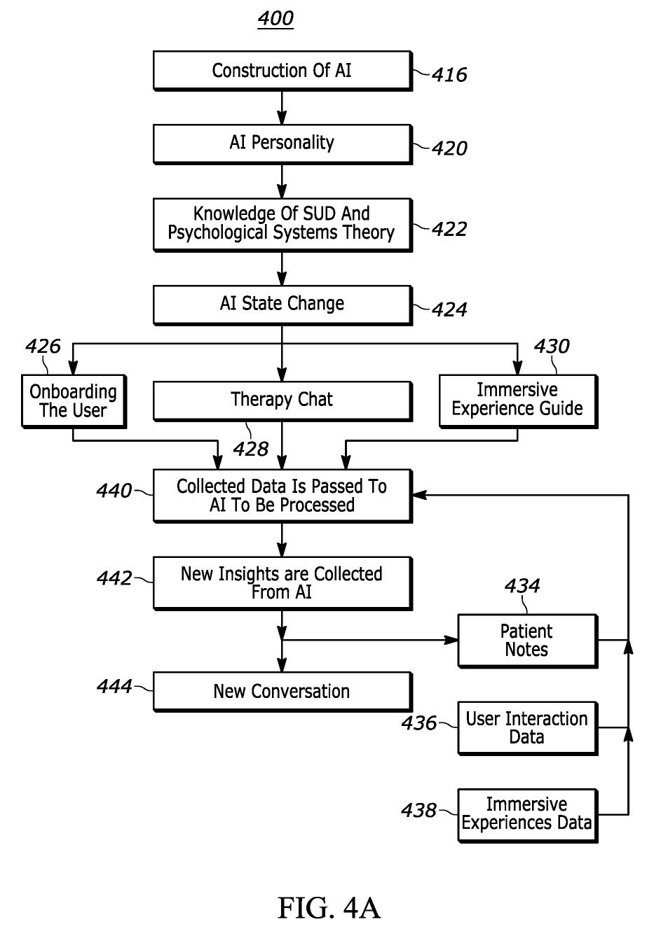

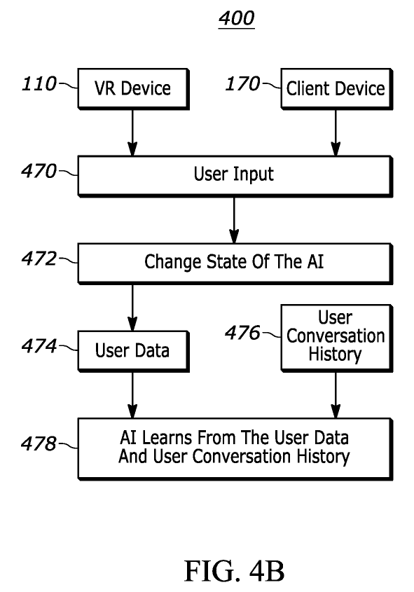

FIGS. 4A and 4B illustrate an exemplary data flow 400 for providing VR therapy.

Referring to FIG. 4A, in one aspect, at block 416 the system constructs the AI model, which may include the generative AI model 144, the therapeutic model 146, and/or the dynamic session builder model 148. Constructing the AI model may include programming a custom AI model or using a pre-built AI model.

In one aspect, at block 420 the personality of the AI model is defined. Defining the personality of the AI model may include implementing high level rules, e.g., how to respond to user suggestions of self-harm, avoiding criticism or negative feedback, etc. Defining the personality of the AI model may include specifying the tone and/or gender of the AI model’s voice output. Defining the personality of the AI model may include specifying one or more high level goals, such as convincing users that they can overcome SUD. Defining the personality of the AI model may include generally specifying how the AI model to respond to user questions or comments.

In one aspect, at block 422 the AI model is trained with knowledge of SUD and Psychological Systems Theory and/or Mindfulness Based Cognitive Therapy. The training data may include definitions, goals, rules, case studies, etc. After training, the AI model may be ready for testing or deployment.

In one aspect, at block 424 the AI model may change state among onboarding the user, therapy chat, and/or immersive experience guide states. The state change may be directed by the user and/or a therapist using a voice command and/or other input. For example, the therapy app 140 context may change from onboarding to chat to immersive experiences, and the AI model changes state accordingly to receive different user inputs and generate different outputs.

In one aspect, at block 426 the AI model onboards the user. Onboarding the user may include the AI model asking for the user’s name, how he or she is feeling, and/or what his or her overall goals are. Onboarding may set the initial tone for future therapy sessions.

In one aspect, at block 428 the AI model engages in therapy chat. In therapy chat, the AI model may engage in interactive dialogue with the user. The therapy chat may include greeting the user, asking the user how he or she is feeling, asking the user for his or her goals for this session, etc. Over the course of one or more sessions, the AI model may ask the user between ten and one hundred questions. During the therapy chat, the AI model may identify key psychological roots of the user. The AI model may generate quantitative metrics based upon the user’s responses and perform statistical analysis upon those metrics. For example, the AI model may apply Pearson product-moment correlational analysis to measure a linear correlation between two sets of data. The AI model may perform multiple regression to identify and/or prioritize the top, e.g., three, key psychological roots.

In one aspect, at block 430 the AI model provides an immersive experience to the user. The immersive experience may be selected from a pre-generated set and/or customized for the user.

In one aspect, at block 434 patient notes are retrieved. The patient notes may include notes written by a physician and/or another therapist. The patient notes may include insights and/or other data generated by the AI model.

In one aspect, at block 436 user interaction data is retrieved. The user interaction data may include user ratings or feedback on interactions, the immersive experiences chosen by the user, how the user performed in the immersive experiences, biometric data, etc. In one aspect, at block 438 immersive experiences data is retrieved. The immersive experiences data may include sounds and colors generated, the user’s game play speed, features that were added or removed by the AI model, etc.

In one aspect, at block 440 data collected from onboarding the user, therapy chat, immersive experience, patient notes, user interaction data, and/or immersive experiences data is passed to the AI model to be processed. In one aspect, at block 442 new insights are collected from the AI model. The insights may include session summaries, diagnoses, recommended treatments, etc.

In one aspect, at block 444 the new insights are passed to the AI model to generate a new conversation. The new conversation may include additional topics or questions based upon the new insights.

Referring now to FIG. 4B, exemplary reinforcement and/or fine-tuning of the AI model is illustrated. In one aspect, a user provides user input at block 470 via the VR device 110 or client device 170. The user input may be typed or spoken and subsequently converted into text. The user input may natural language dialogue by the user engaging in a conversation or providing short answers to questions.

In one aspect, the system at block 472 changes the state of the AI model. The state change may be based on a context change of the therapy app 140.

In one aspect, at block 474 the AI model receives user data. The user data may be received via the therapy app 140 during the onboarding state. The user data may be subsequently retrieved from data storage 156. The user data may include the user’s name, symptoms, treatment goals, SUD use history, family information, etc.

In one aspect, at block 476 the AI model receives user conversation history. The user conversation history may include a transcript or a summary of conversations during one or more prior user sessions. The user conversation history may include the context in which the conversation occurred, such as onboarding, chat, or immersive experiences.

In one aspect, at block 478 the AI model learns from the user data and/or user conversation history. The user data and/or user conversation history may be added to training data 210 and used by the machine learning module 150 to fine-tune the generative AI model 144.

### EXEMPLARY COMPUTER-IMPLEMENTED METHOD FOR PROVIDING VR THERAPY

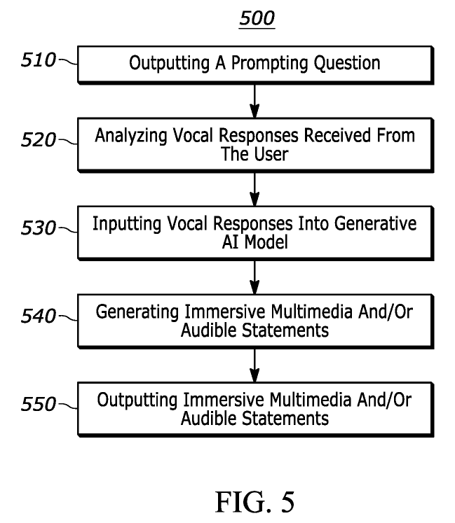

FIG. 5 illustrates a flow diagram of an exemplary computer-implemented method 500 for providing therapeutic, user-specific treatment for SUD. One or more steps of the computer-implemented method 500 may be implemented as a set of instructions stored on a computer-readable memory and executable on one or more processors. The computer-implemented method 500 of FIG. 5 may be implemented via a system, such as the VR device 110, therapy server 130, biometric sensors 160, and/or the client device 170.

In one aspect, the computer-implemented method 500 may include at block 510 outputting, by a voice assistant of an audible output device of a VR device, a user prompt configured to trigger a dynamic session builder with a user experiencing SUD. The SUD may be an OUD. The user prompt may be a user-specific statement or question based on the user-specific data of the user or the one or more existing dynamic sessions with the user. The user prompt may be generated and output to continue at least one of the one or more existing dynamic sessions with the user. The VR device may include a display screen positioned proximate to, or within a viewable distance from, eyes of the user. The VR device may be communicatively coupled to one or more processors and one or more input devices. The audible output device may be communicatively connected to the one or more processors of the VR device. The voice assistant may be accessible by the one or more processors. The voice assistant may include a generative AI model. The generative AI model may be trained with training data indicative of one or more psychological causes or psychological triggers of SUD. The generative AI model may be further trained with a plurality of immersive multimedia scenes designed to treat respective urges or cravings corresponding to the one or more psychological causes or psychological triggers of SUD. The generative AI model may be trained on user-specific data of the user. The generative AI model may be trained on one or more existing dynamic sessions with the user.

In one aspect, the computer-implemented method 500 may include at block 520 analyzing, by the voice assistant, one or more vocal responses received from the user having user data. The vocal responses may be indicative of a user-specific psychological cause or psychological trigger of the user causing an SUD of the user.

In one aspect, the computer-implemented method 500 may include at block 530 inputting, by the one or more processors, the one or more vocal responses into the generative AI model.

In one aspect, the computer-implemented method 500 may include at block 540 generating, by the generative AI model based on the one or more vocal responses of the user received during the dynamic session builder, one or more immersive multimedia scenes and/or one or more audible statements. Generating the one or more immersive multimedia scenes and/or the one or more audible statements may be further based on biometric data of the user. The immersive multimedia scenes and/or audible statements may be designed to reduce urges or cravings corresponding to the user-specific psychological cause or psychological trigger of the user. The immersive multimedia scenes may depict a 3D room. The 3D room may be customized for the user based on the one or more existing dynamic sessions of the user. The 3D room may include selections or areas in 3D space for beginning execution of immersive experiences for implementing the therapeutic treatment for the user’s SUD. The immersive multimedia scenes may depict a visualization of the breath of the user comprising particle simulations. The particle simulations may mimic a flow of air during inhalation and exhalation by the user. The dynamic session builder may be an immersive experience type. The immersive experience type may cause the generative AI model to generate the immersive multimedia scenes and/or the statements according to a preselected immersive experience theme, such as a therapy session-based theme, a creative-based theme, an action play motion theme, a meditation-based theme, an exploratory theme, a problem-solving skill building theme, or an interpersonal relationship building theme.

In one aspect, the computer-implemented method 500 may include at block 550 outputting, by the display screen the one or more immersive scenes, outputting, by the audible output device, the one or more audible statements, and/or outputting, by a haptic feedback device, haptic feedback to the user as a therapeutic treatment for the SUD.

In one aspect, the computer-implemented method 500 may include outputting one or more results of the therapeutic treatment of the user’s SUD. The one or more results may include one or more of a graphic, chart, or data of the biometric data of the user and a survey by the user generated by collecting additional vocal responses of the user and/or by collecting input from the user operating the one or more input devices.

It should be understood that not all blocks of the exemplary computer-implemented method 500 are required to be performed. Moreover, the exemplary computer-implemented method 500 is not mutually exclusive (i.e., block(s) from the exemplary computer-implemented method 500 may be performed in any particular implementation).

### ADDITIONAL CONSIDERATIONS

Although the text herein sets forth a detailed description of numerous different embodiments, it should be understood that the legal scope of the invention is defined by the words of the claims set forth at the end of this patent. The detailed description is to be construed as exemplary only and does not describe every possible embodiment, as describing every possible embodiment would be impractical, if not impossible. One could implement numerous alternate embodiments, using either current technology or technology developed after the filing date of this patent, which would still fall within the scope of the claims.

It should also be understood that, unless a term is expressly defined in this patent using the sentence “As used herein, the term ‘XYZ’ is hereby defined to mean…” or a similar sentence, there is no intent to limit the meaning of that term, either expressly or by implication, beyond its plain or ordinary meaning, and such term should not be interpreted to be limited in scope based upon any statement made in any section of this patent (other than the language of the claims). To the extent that any term recited in the claims at the end of this disclosure is referred to in this disclosure in a manner consistent with a single meaning, that is done for sake of clarity only so as to not confuse the reader, and it is not intended that such claim term be limited, by implication or otherwise, to that single meaning. Finally, unless a claim element is defined by reciting the word “means” and a function without the recital of any structure, it is not intended that the scope of any claim element be interpreted based upon the application of 35 U.S.C. § 112(f).

Throughout this specification, plural instances may implement components, operations, or structures described as a single instance. Although individual operations of one or more methods are illustrated and described as separate operations, one or more of the individual operations may be performed concurrently, and nothing requires that the operations be performed in the order illustrated. Structures and functionality presented as separate components in exemplary configurations may be implemented as a combined structure or component. Similarly, structures and functionality presented as a single component may be implemented as separate components. These and other variations, modifications, additions, and improvements fall within the scope of the subject matter herein.

Additionally, certain embodiments are described herein as including logic or a number of routines, subroutines, applications, or instructions. These may constitute either software (code embodied on a non-transitory, tangible machine-readable medium) or hardware. In hardware, the routines, etc., are tangible units capable of performing certain operations and may be configured or arranged in a certain manner. In exemplary embodiments, one or more computer systems (e.g., a standalone, client or server computer system) or one or more hardware modules of a computer system (e.g., a processor or a group of processors) may be configured by software (e.g., an application or application portion) as a hardware module that operates to perform certain operations as described herein.

In various embodiments, a hardware module may be implemented mechanically or electronically. For example, a hardware module may comprise dedicated circuitry or logic that is permanently configured (e.g., as a special-purpose processor, such as a field programmable gate array (FPGA) or an application-specific integrated circuit (ASIC) to perform certain operations). A hardware module may also comprise programmable logic or circuitry (e.g., as encompassed within a processor or other programmable processor) that is temporarily configured by software to perform certain operations. It will be appreciated that the decision to implement a hardware module mechanically, in dedicated and permanently configured circuitry, or in temporarily configured circuitry (e.g., configured by software) may be driven by cost and time considerations.

Accordingly, the term “hardware module” should be understood to encompass a tangible entity, be that an entity that is physically constructed, permanently configured (e.g., hardwired), or temporarily configured (e.g., programmed) to operate in a certain manner or to perform certain operations described herein. Considering embodiments in which hardware modules are temporarily configured (e.g., programmed), each of the hardware modules need not be configured or instantiated at any one instance in time. For example, where the hardware modules comprise a processor configured using software, the processor may be configured as respective different hardware modules at different times. Software may accordingly configure a processor, for example, to constitute a particular hardware module at one instance of time and to constitute a different hardware module at a different instance of time.

Hardware modules can provide information to, and receive information from, other hardware modules. Accordingly, the described hardware modules may be regarded as being communicatively coupled. Where multiple of such hardware modules exist contemporaneously, communications may be achieved through signal transmission (e.g., over appropriate circuits and buses) that connect the hardware modules. In embodiments in which multiple hardware modules are configured or instantiated at different times, communications between such hardware modules may be achieved, for example, through the storage and retrieval of information in memory structures to which the multiple hardware modules have access. For example, one hardware module may perform an operation and store the output of that operation in a memory device to which it is communicatively coupled. A further hardware module may then, at a later time, access the memory device to retrieve and process the stored output. Hardware modules may also initiate communications with input or output devices, and can operate on a resource (e.g., a collection of information).

The various operations of exemplary methods described herein may be performed, at least partially, by one or more processors that are temporarily configured (e.g., by software) or permanently configured to perform the relevant operations. Whether temporarily or permanently configured, such processors may constitute processor-implemented modules that operate to perform one or more operations or functions. The modules referred to herein may, in some exemplary embodiments, comprise processor-implemented modules.

Similarly, the methods or routines described herein may be at least partially processor-implemented. For example, at least some of the operations of a method may be performed by one or more processors or processor-implemented hardware modules. The performance of certain of the operations may be distributed among the one or more processors, not only residing within a single machine, but deployed across a number of machines. In some example embodiments, the processor or processors may be located in a single location (e.g., within a home environment, an office environment or as a server farm), while in other embodiments the processors may be distributed across a number of geographic locations.

Unless specifically stated otherwise, discussions herein using words such as processing,” “computing,” “calculating,” “determining,” “presenting,” “displaying,” or the like may refer to actions or processes of a machine (e.g., a computer) that manipulates or transforms data represented as physical (e.g., electronic, magnetic, or optical) quantities within one or more memories (e.g., volatile memory, non-volatile memory, or a combination thereof), registers, or other machine components that receive, store, transmit, or display information.

As used herein any reference to “one embodiment” or “an embodiment” means that a particular element, feature, structure, or characteristic described in connection with the embodiment may be included in at least one embodiment. The appearances of the phrase “in one embodiment” in various places in the specification are not necessarily all referring to the same embodiment.

Some embodiments may be described using the expression “coupled” and “connected” along with their derivatives. For example, some embodiments may be described using the term “coupled” to indicate that two or more elements are in direct physical or electrical contact. The term “coupled,” however, may also mean that two or more elements are not in direct contact with each other, but yet still co-operate or interact with each other. The embodiments are not limited in this context.

As used herein, the terms “comprises,” “comprising,” “includes,” “including,” “has,” “having” or any other variation thereof, are intended to cover a non-exclusive inclusion. For example, a process, method, article, or apparatus that comprises a list of elements is not necessarily limited to only those elements but may include other elements not expressly listed or inherent to such process, method, article, or apparatus. Further, unless expressly stated to the contrary, “or” refers to an inclusive or and not to an exclusive or. For example, a condition A or B is satisfied by any one of the following: A is true (or present) and B is false (or not present), A is false (or not present) and B is true (or present), and both A and B are true (or present).

In addition, use of the “a” or “an” are employed to describe elements and components of the embodiments herein. This is done merely for convenience and to give a general sense of the description. This description, and the claims that follow, should be read to include one or at least one and the singular also includes the plural unless it is obvious that it is meant otherwise.

Upon reading this disclosure, those of skill in the art will appreciate still additional alternative structural and functional designs for the approaches described herein. Therefore, while particular embodiments and applications have been illustrated and described, it is to be understood that the disclosed embodiments are not limited to the precise construction and components disclosed herein. Various modifications, changes and variations, which will be apparent to those skilled in the art, may be made in the arrangement, operation and details of the method and apparatus disclosed herein without departing from the spirit and scope defined in the appended claims.

The particular features, structures, or characteristics of any specific embodiment may be combined in any suitable manner and in any suitable combination with one or more other embodiments, including the use of selected features without corresponding use of other features. In addition, many modifications may be made to adapt a particular application, situation or material to the essential scope and spirit of the present invention. It is to be understood that other variations and modifications of the embodiments of the present invention described and illustrated herein are possible in light of the teachings herein and are to be considered part of the spirit and scope of the present invention.

While the preferred embodiments of the invention have been described, it should be understood that the invention is not so limited, and modifications may be made without departing from the invention. The scope of the invention is defined by the appended claims, and all devices that come within the meaning of the claims, either literally or by equivalence, are intended to be embraced therein.

It is therefore intended that the foregoing detailed description be regarded as illustrative rather than limiting, and that it be understood that it is the following claims, including all equivalents, that are intended to define the spirit and scope of this invention.

What is claimed is:

1. A virtual reality (VR) system configured to provide therapeutic, user-specific treatment for substance use disorder (SUD):
a VR device comprising a display screen positioned proximate to, or within a viewable distance from, eyes of a user experiencing SUD, the VR device communicatively coupled to one or more processors and one or more input devices;
an audible output device communicatively connected to the one or more processors;
a voice assistant accessible by the one or more processors;
a generative artificial intelligence (AI) model trained to identify user-specific psychological roots of SUD based on vocal input and generate a dynamic session prompt comprising a description of an immersive multimedia experience created based on the identified user-specific psychological roots, wherein the psychological roots comprise underlying cognitive or emotional cues associated with substance-related cravings, wherein the generative AI model is trained with training data indicative of the psychological roots and further trained with a plurality of immersive multimedia scenes designed to treat respective urges or cravings corresponding to one or more psychological causes or psychological triggers of SUD; and
an application (app) comprising computing instructions stored on a memory communicatively coupled to the one or more processors, wherein the computing instructions, when executed by the one or more processors, are configured to cause the one or more processors to:
output, by the voice assistant through the audible output device, a user prompt configured to trigger a dynamic session builder with the user,
analyze, by the voice assistant, one or more vocal responses received from the user having user data indicative of a user-specific psychological cause or psychological trigger of the user causing SUD,
input, into the generative AI model, the one or more vocal responses,
generate, by the generative AI model based on the one or more vocal responses received during the dynamic session builder, one or more immersive multimedia scenes and/or one or more audible statements designed to reduce urges or cravings corresponding to the user-specific psychological cause or psychological trigger of the user, and
output the one or more immersive multimedia scenes on the display screen and/or the one or more audible statements via the audible output device, to the user as a therapeutic treatment for SUD.

2. The VR system of claim 1, wherein the dynamic session builder coordinates user sessions based on session transcripts, audio, user biometric data, and/or user feedback from one or more users, and wherein the one or more immersive multimedia scenes and/or one or more audible statements are generated based on the user sessions.

3. The VR system of claim 2, wherein at least one of the one or more immersive multimedia scenes depict the one or more users in a three-dimensional (3D) room or space, each of the one or more users providing content that addresses a respective identified psychological root of the user-specific psychological roots.

4. The VR system of claim 1 further comprising one or more sensors in contact with the user and configured to collect biometric data associated with a physiological state of the user, and wherein generation of the one or more immersive multimedia scenes and/or the one or more audible statements is further based on the biometric data of the user.

5. The VR system of claim 4, wherein the one or more sensors comprises at least one of: (a) an electroencephalography (EEG) sensor; or (b) a biometric measurement sensor, wherein the biometric measurement sensor is part of a wearable device.

6. The VR system of claim 1 further comprising:
a haptic feedback device,
and wherein the computing instructions, when executed by the one or more processors, are further configured to cause the one or more processors to:
output haptic feedback via the haptic feedback device during display of the one or more immersive multimedia scenes on the display screen and/or during output of the one or more audible statements via the audible output device.

7. The VR system of claim 1, wherein the training data upon which the generative AI model is trained is user-specific data of the user.

8. The VR system of claim 7, wherein the training data upon which the generative AI model is trained on comprises one or more existing dynamic sessions with the user.

9. The VR system of claim 8, wherein the user prompt is a user-specific statement or question based on the user-specific data of the user or the one or more existing dynamic sessions with the user, and wherein the user prompt is generated and output to continue at least one of the one or more existing dynamic sessions with the user.

10. The VR system of claim 8, wherein the one or more immersive multimedia scenes depict a three-dimensional (3D) room, and wherein the 3D room is customized for the user based on the one or more existing dynamic sessions of the user, and wherein the 3D room includes selections or areas in 3D space for beginning execution of immersive experiences for implementing the therapeutic treatment for SUD.

11. The VR system of claim 1, wherein the one or more immersive multimedia scenes depict a visualization of breath of the user comprising particle simulations, wherein the particle simulations mimic a flow of air during inhalation and exhalation by the user.

12. The VR system of claim 1, wherein the dynamic session builder comprises a immersive experience type, the immersive experience type causing the generative AI model to generate the one or more immersive multimedia scenes and/or the one or more audible statements according to a preselected immersive experience theme selected from one of: (a) a therapy session based theme; (b) a creative based theme; (c) an action play motion theme; (d) a meditation based theme; (e) an exploratory theme; (f) a problem-solving skill building theme; or (g) an interpersonal relationship building theme.

13. The VR system of claim 3, wherein the computing instructions, when executed by the one or more processors, are further configured to cause the one or more processors to:
output one or more results of the therapeutic treatment of SUD, the one or more results comprising at least one of: (a) a graphic, chart, or data of the biometric data of the user; or (b) a survey by the user generated by collecting additional vocal responses of the user and/or by collecting input from the user operating the one or more input devices.

14. The VR based system of claim 1, wherein the psychological roots comprise at least one of: (a) the user’s underlying belief system; or (b) the user’s underlying goals.

15. A virtual reality (VR) based method for providing therapeutic, user-specific treatment for substance use disorder (SUD):
outputting, by a voice assistant communicatively connected to an audible output device of a VR device, a user prompt configured to trigger a dynamic session builder with a user experiencing SUD,
wherein the VR device comprises a display screen positioned proximate to, or within a viewable distance from, eyes of the user, the VR device communicatively coupled to one or more processors and one or more input devices,
the audible output device communicatively connected to the one or more processors, and
inputting, by the voice assistant, one or more vocal responses received from the user having user data indicative of a user-specific psychological cause or psychological trigger of the user causing an SUD of the user;
inputting, by the one or more processors, the one or more vocal responses into a generative artificial intelligence (AI) model, wherein the generative AI model is trained to identify user-specific psychological roots of SUD based on vocal input and generate a dynamic session prompt comprising a description of an immersive multimedia experience created based on the identified user-specific psychological roots, wherein psychological roots comprise underlying cognitive or emotional cues associated with substance-related cravings, wherein the generative AI model is trained with training data indicative of the psychological roots and further trained with a plurality of immersive multimedia scenes designed to treat respective urges or cravings corresponding to one or more psychological causes or psychological triggers of SUD;
generating, by the generative AI model based on the one or more vocal responses of the user received during the dynamic session builder, one or more immersive multimedia scenes and/or one or more audible statements designed to reduce urges or cravings corresponding to the user-specific psychological cause or psychological trigger of the user; and
outputting, by the display screen and/or the audible output device, the one or more immersive multimedia scenes and/or the one or more audible statements, to the user as a therapeutic treatment for SUD.

16. The VR based method of claim 15, wherein generation of the one or more immersive multimedia scenes and/or the one or more audible statements is further based on biometric data associated with a physiological state of the user.

17. The VR based method of claim 15, wherein the training data upon which the generative AI model is trained on comprises one or more existing dynamic sessions with the user.

18. The VR based method of claim 17, wherein the user prompt is a user-specific statement or question based on the user-specific data of the user or the one or more existing dynamic sessions with the user, and wherein the user prompt is generated and output to continue at least one of the one or more existing dynamic sessions with the user.

19. The VR based method of claim 17, wherein the one or more immersive multimedia scenes depict a three-dimensional (3D) room, and wherein the 3D room is customized for the user based on the one or more existing dynamic sessions of the user, and wherein the 3D room includes selections or areas in 3D space for beginning execution of immersive experiences for implementing the therapeutic treatment for SUD.

20. The VR based method of claim 15, wherein the dynamic session builder comprises a immersive experience type, the immersive experience type causing the generative AI model to generate the one or more immersive multimedia scenes and/or the one or more audible statements according to a preselected immersive experience theme selected from one of: (a) a therapy session based theme; (b) a creative based theme; (c) an action play motion theme; (d) a meditation based theme; (e) an exploratory theme; (f) a problem-solving skill building theme; or (g) an interpersonal relationship building theme.
